(19) United States
(12) Patent Application Publication
(10) Pub. No.: US 2020/0337539 A1
(43) Pub. Date: Oct. 29, 2020

(54) IMAGE PICKUP APPARATUS, ENDOSCOPE, AND METHOD OF MANUFACTURING IMAGE PICKUP APPARATUS

(71) Applicant: OLYMPUS CORPORATION, Tokyo (JP)

(72) Inventors: Takahiro SHIMOHATA, Shiojiri-shi (JP); Kensuke SUGA, Kamiina-gun (JP); Hiroyuki MOTOHARA, Tokyo (JP)

(73) Assignee: OLYMPUS CORPORATION, Tokyo (JP)

(21) Appl. No.: 16/925,468

(22) Filed: Jul. 10, 2020

Related U.S. Application Data
(63) Continuation of application No. PCT/JP2018/000285, filed on Jan. 10, 2018.

(51) Int. Cl. A61B 1/05; A61B 1/00; H01L 27/146

(52) U.S. Cl. CPC A61B 1/051; A61B 1/00114; H01L 27/14687; H01L 27/14634; H01L 27/14636; H01L 27/14618

(57) ABSTRACT

An image pickup apparatus disposed in an endoscope includes: an image pickup unit including an image pickup device, a stacked element and a plurality of rear electrodes; an interposer having a recessed portion in which the image pickup unit is housed, wherein a plurality of connection electrodes which are connected with the plurality of rear electrodes are disposed on a bottom surface of the recessed portion, and a plurality of bonding electrodes which are connected with the plurality of connection electrodes are disposed on an outer surface of the interposer; a sealing resin; and a plurality of electric cables connected to the plurality of bonding electrodes of the interposer. An arrangement interval between the plurality of bonding electrodes is set larger than an arrangement interval between the plurality of connection electrodes.

### CROSS REFERENCE TO RELATED APPLICATION

This application is a continuation application of PCT/JP2018/000285 filed on Jan. 10, 2018, the entire contents of which are incorporated herein by this reference.

### BACKGROUND OF THE INVENTION

### 1. Field of the Invention

The present invention relates to an image pickup apparatus including a stacked element formed by stacking a plurality of semiconductor devices and disposed on a distal end portion of an endoscope, an endoscope having a distal end portion on which an image pickup apparatus including a stacked element formed by stacking a plurality of semiconductor devices is disposed, and a method of manufacturing an image pickup apparatus including a stacked element formed by stacking a plurality of semiconductor devices and disposed on a distal end portion of air endoscope.

### 2. Description of the Related Art

An image pickup signal which an image pickup device disposed on a distal end portion of an endoscope outputs is processed through primary processing by a plurality of electronic components and the processed signal is transmitted thereafter.

For example, Japanese Patent Application Laid-Open Publication No. 2005-334509 discloses an endoscope having an image pickup apparatus which transmits an image pickup signal which is processed through primary processing by a plurality of electronic components mounted on a wiring board by an electric cable.

Japanese Patent Application Laid-Open Publication No. 2013-30593 discloses a stacked element which is formed by stacking a plurality of semiconductor devices for housing a plurality of semiconductor devices in a small space and for reducing a parasitic capacitance generated by wiring.

International Publication No. 2017/073440 discloses an endoscope which realizes downsizing and sophistication of functions of an image pickup apparatus by using a stacked element.

An electric cable or a wiring board is connected to an image pickup apparatus for transmitting an image pickup signal or for supplying a drive power source to the image pickup apparatus. In the image pickup apparatus which includes a stacked element, the electric cable or the like and the stacked element are bonded to each other by soldering. The image pickup apparatus to which the electric cable or the like is connected is disposed on a distal end portion of an endoscope in an assembling step.

### SUMMARY OF THE INVENTION

An image pickup apparatus according to an embodiment is disposed in an endoscope. The image pickup apparatus includes: an image pickup unit including an image pickup device, a stacked element formed by stacking a plurality of semiconductor devices, and a plurality of rear electrodes; an interposer having a recessed portion in which the image pickup unit is housed, wherein a plurality of connection electrodes which are respectively connected with the plurality of rear electrodes are disposed on a bottom surface of the recessed portion, and a plurality of bonding electrodes which are respectively connected with the plurality of connection electrodes are disposed on an outer surface of the interposer; a sealing resin disposed between the image pickup unit and the interposer; and a plurality of electric cables or a wiring board respectively connected to the plurality of bonding electrodes of the interposer. An arrangement interval between the plurality of bonding electrodes is set larger than an arrangement interval between the plurality of connection electrodes.

An endoscope according to an embodiment includes an image pickup apparatus. The image pickup apparatus is disposed in the endoscope. The image pickup apparatus includes: an image pickup unit including an image pickup device, a stacked element formed by stacking a plurality of semiconductor devices, and a plurality of rear electrodes; an interposer having a recessed portion in which the image pickup unit is housed, wherein a plurality of connection electrodes which are respectively connected with the plurality of rear electrodes are disposed on a bottom surface of the recessed portion, and a plurality of bonding electrodes which are respectively connected with the plurality of connection electrodes are disposed on an outer surface of the interposer; a sealing resin disposed between the image pickup unit and the interposer; and a plurality of electric cables or a wiring board respectively connected to the plurality of bonding electrodes of the interposer. An arrangement interval between the plurality of bonding electrodes is set larger than an arrangement interval between the plurality of connection electrodes.

A method of manufacturing an image pickup apparatus according to the embodiment includes: preparing an image pickup unit including an image pickup device, a stacked element formed by stacking a plurality of semiconductor devices, and a plurality of rear electrodes; preparing an interposer having a recessed portion, wherein a plurality of connection electrodes are disposed on a bottom surface of the recessed portion, and a plurality of bonding electrodes which are respectively connected with the plurality of connection electrodes are disposed on an outer surface of the interposer; housing the image pickup unit in the recessed portion of the interposer, and connecting the respective plurality of rear electrodes with the respective plurality of connection electrodes; disposing a resin between the image pickup unit and the interposer; and connecting a respective plurality of electric cables or a wiring board to the plurality of bonding electrodes of the interposer, wherein an arrangement interval between the plurality of bonding electrodes is set larger than an arrangement interval between the plurality of connection electrodes.

### BRIEF DESCRIPTION OF THE DRAWINGS

FIG. 1 is a perspective view of an endoscope according to an embodiment;

FIG. 2 is a perspective view of an image pickup apparatus according to a first embodiment;

FIG. 3 is a cross-sectional view of the image pickup apparatus according to the first embodiment taken along a line III-III in FIG. 2;

FIG. 4 is an exploded view of the image pickup apparatus according to the first embodiment;

FIG. 5 is a flowchart of a method of manufacturing an image pickup apparatus according to the first embodiment;

FIG. 6 is a cross-sectional view of an image pickup apparatus according to a second embodiment;

FIG. 7 is a cross-sectional view of an image pickup apparatus according to a modification 1 of the second embodiment;

FIG. 8 is a cross-sectional view of an image pickup apparatus according to a modification 2 of the second embodiment;

FIG. 9 is a perspective view of an image pickup apparatus according to a modification 3 of the second embodiment;

FIG. 10 is a perspective view of an interposer of an image pickup apparatus according to a modification 4 of the second embodiment;

FIG. 11 is a perspective view of an interposer of an image pickup apparatus according to a modification 5 of the second embodiment;

FIG. 12 is a perspective view of an interposer of an image pickup apparatus according to a modification 6 of the second embodiment;

FIG. 13 is a perspective view of an interposer of an image pickup apparatus according to a modification 7 of the second embodiment; and

FIG. 14 is a perspective view of an interposer of an image pickup apparatus according to a modification 8 of the second embodiment.

### DETAILED DESCRIPTION OF THE PREFERRED EMBODIMENTS

### First Embodiment

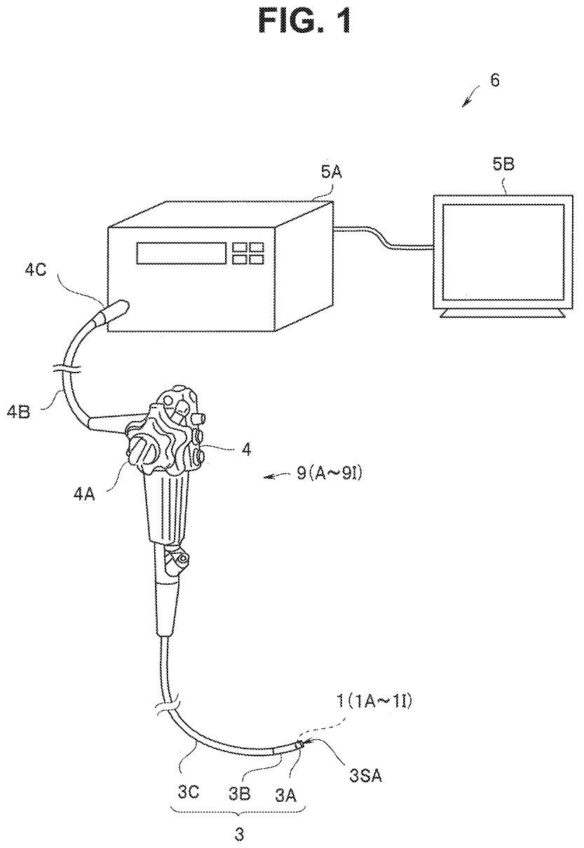

As shown in FIG. 1, an image pickup apparatus 1 according to an embodiment is disposed at a distal end portion 3A of an insertion section 3 of an endoscope 9.

The endoscope 9 includes: the insertion section 3; a grasping section 4 disposed on a proximal end portion side of the insertion section 3; a universal cord 4B extending from the grasping section 4; and a connector 4C disposed on a proximal end portion side of the universal cord 4B. The insertion section 3 includes: a distal end portion 3A on which the image pickup apparatus 1 is disposed; a bending portion 3B extending toward a proximal end side of the distal end portion 3A and being bendable for changing a direction of the distal end portion 3A; and a flexible portion 3C extending toward a proximal end side of the bending portion 3B. A rotatable angle knob 4A which is an operation portion operated by a surgeon for operating the bending portion 3B is disposed on the grasping section 4.

The universal cord 4B is connected to a processor 5A via the connector 4C. The processor 5A controls an entire endoscope system 6, and applies signal processing to an image pickup signal outputted from the image pickup apparatus 1, and outputs the processed signal as an image signal. A monitor 5B displays the image signal which the processor 5A outputs as an endoscope image. Although the endoscope 9 is a flexible endoscope in the embodiment, an endoscope according to the present invention may be a rigid endoscope, and is used both in a medical field and in an industrial field.

### Configuration of Image Pickup Apparatus

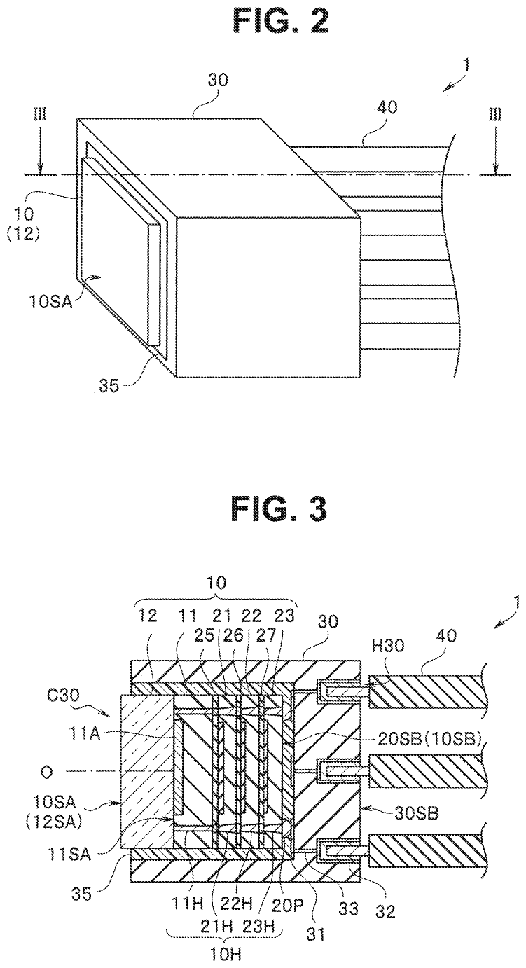

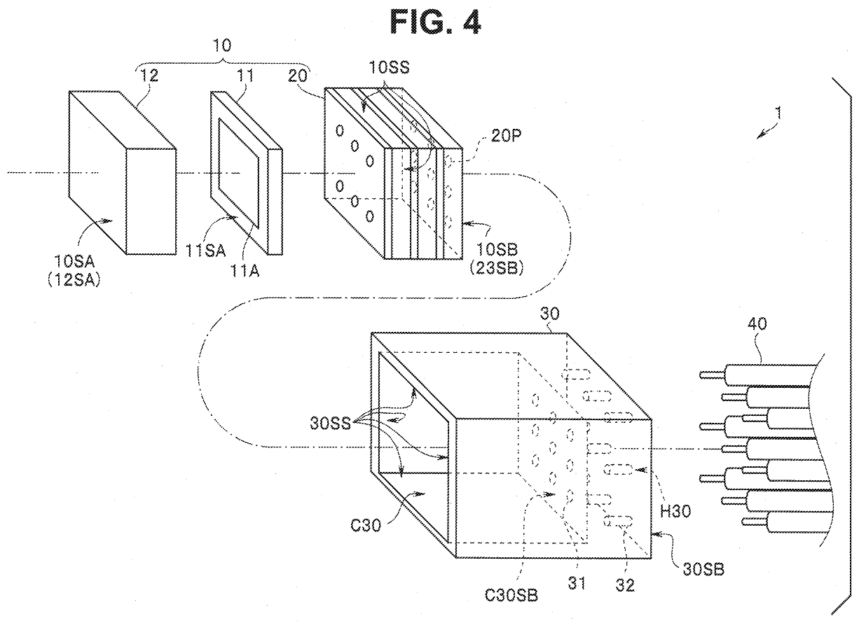

As shown in FIG. 2 to FIG. 4, the image pickup apparatus 1 includes an image pickup unit 10; an interposer 30, a sealing resin 35, and a plurality of electric cables 40. The image pickup apparatus 1 receives image pickup light which is light converged by an optical unit not shown, converts the image pickup light into an electric signal, and outputs the electric signal to the processor 5A via a plurality of electric cables 40 after applying primary processing to the electric signal.

Drawings based on the respective embodiments are schematic views. Accordingly, it should be noted that a relationship between a thickness and a width of each member, a ratio between thicknesses, a relative angle and the like of respective members, differ from the corresponding relationships of members of an actual endoscope. There may be a case where portions of the endoscope are described with different size relationship or different ratios between the drawings. There may also be a case where some constitutional components are not illustrated or symbols are not given to some constitutional components.

The image pickup unit 10 includes a cover glass 12, an image pickup device 11, a stacked element 20 formed by stacking a plurality of semiconductor devices 21, 22, 23. The image pickup device 11 and the like are parallel planar chips, and the image pickup unit 10 is a rectangular parallelepiped body which has: a front surface 10SA (12SA); a rear surface 10SB (23SB) disposed on a side opposite to the front surface 10SA; and four side surfaces 10SS.

The image pickup device 11 has a light receiving portion 11A formed of a CCD or CMOS image pickup portion, and the light receiving portion 11A is connected with penetrating wires 11H. The image pickup device 11 may be either a front-illuminated image sensor or a back-illuminated image sensor.

The cover glass 12 which covers and protects the light receiving portion 11A adheres to a light receiving surface 11SA of the image pickup device 11 using an adhesive layer (not shown). Although the cover glass 12 protects the light receiving portion 11A in manufacturing steps, the cover glass 12 is not an indispensable constitutional component for forming the image pickup unit 10. The cover glass 12 is not limited to a parallel planar chip made of glass, and may be a resin plate, a ceramic plate or the like having high optical transmissivity with respect to an image pickup light.

The semiconductor devices 21 to 23 which form the stacked element 20 respectively have penetrating wires 21H to 23H, and the semiconductor devices 21 to 23 are electrically connected with each other. The image pickup device 11 and the semiconductor devices 21 to 23 are connected to each other by a solder bump formed by an electroplating method or by a solder bonding portion made of a solder paste film formed by printing or the like.

A sealing resin 25 is disposed between the image pickup device 11 and the semiconductor device 21, a sealing resin 26 is disposed between the semiconductor device 21 and the semiconductor device 22, and a sealing resin 27 is disposed between the semiconductor device 22 and the semiconductor device 23. The stacked element 20 applies primary processing to an image pickup signal outputted from the image pickup device 11, or applies processing to a control signal controlling the image pickup device 11. For example, the semiconductor devices 21 to 23 include an AD convertor, a memory, a transmission output circuit, a filter circuit, a thin film capacitor, a thin film inductor and the like. The number of devices which the image pickup unit 10 including the image pickup device 11 has is 3 to 10 inclusive, for example. The image pickup apparatus 1 which includes the stacked element 20 is small-sized and has high functions.

A plurality of rear electrodes 20P are disposed on the rear surface 10SB of the image pickup unit 10 (a rear surface 23SB of the semiconductor device 23 which is stacked on a rearmost portion). The rear electrode 20P is, for example, a convex electrode which is disposed on a wiring pattern made of Cu, and is formed of a barrier Ni layer and an Au layer.

The interposer 30 is disposed between the image pickup unit 10 and the plurality of electric cables 40, and electrically connects the image pickup unit 10 and the plurality of electric cables 40 to each other. The interposer is an MID (molded interconnect device) or a ceramic stereoscopic substrate. When the plurality of electric cables 40 are directly bonded to the image pickup unit 10, a mechanical load and a thermal load are applied to the image pickup unit 10 and hence, there is a risk that reliability of the image pickup apparatus 1 is lowered. By connecting the image pickup unit 10 and the plurality of electric cables 40 using the interposer 30, the reliability of the image pickup apparatus 1 can be ensured.

The interposer 30 has a function of a protective member which protects the image pickup unit 10 in addition to the previously-mentioned usual interposer function.

A recessed portion (concave portion) C30 in which the image pickup unit 10 is housed is formed in the interposer 30. In other words, an inner size of the recessed portion C30 which has four wall surfaces 30SS opposedly facing four side surfaces 10SS of the image pickup unit 10 and a bottom surface C30SB is larger than an outer size of the image pickup unit 10. Connection electrodes 31 are disposed on the bottom surface C30SB of the recessed portion C30, and bonding electrodes 32 which are connected to the connection electrodes 31 via penetration wires 33 are disposed on an outer surface 30SB which opposedly faces the bottom surface C30SB. Note that the bottom surface C30SB is an opposedly facing surface which opposedly faces the rear surface 10SB of the image pickup unit 10, and the bottom surface C30SB is disposed at the position closer to the stacked element 20 than the image pickup device 11.

The image pickup unit 10 is housed in the recessed portion C30 of the interposer 30, and the rear electrodes 20P of the image pickup unit 10 and the connection electrodes 31 of the interposer 30 are bonded to each other.

The sealing resin 35 is disposed in a gap formed between the image pickup unit 10 and the interposer 30. The sealing resin 35 prevents infiltration of moisture from side surfaces of the image pickup unit 10 thus improving reliability of the image pickup apparatus 1. Further, in the image pickup apparatus 1, the sealing resin 35 also has an effect of alleviating an impact force applied to the image pickup unit 10 when the impact force is applied to the interposer 30.

In the interposer 30, a plurality of holes H30 are formed in the outer surface 30SB disposed opposite to the bottom surface C30SB of the recessed portion C30. Distal end portions of the plurality of electric cables 40 are respectively inserted into and bonded to the plurality of holes H30 each having a wall surface on which the bonding electrode 32 is disposed. With such a configuration, the electric cables 40 can be easily bonded to the interposer 30.

Although the bonding electrodes 32 and the electric cables 40 are bonded to each other by soldering, for example, there is no possibility that a load is applied to the image pickup unit 10 at the time of performing such bonding.

The electric cables 40 may be electrically connected to the interposer 30 with a wiring board sandwiched between the electric cables 40 and the interposer 30. That is, for example, a flexible wiring board is bonded to the bonding electrodes 32 of the interposer 30 by soldering, and the electric cables may be connected to the wiring board.

In the image pickup unit 10, provided that the entirety of the image pickup device 11 and the stacked element 20 is housed in the recessed portion C30 and is protected by the sealing resin 35, a portion of the image pickup unit 10, for example, a portion of the cover glass 12 may protrude from the recessed portion C30.

In the image pickup apparatus 1, the image pickup unit 10 which includes the stacked element 20 having a not so high mechanical strength is housed in the recessed portion C30 of the interposer 30, and the image pickup unit 10 is protected by the sealing resin 35. Accordingly, there is no risk that image pickup apparatus 1 is broken during manufacturing steps, particularly, at the time of bonding the electric cables 40, and reliability of the image pickup apparatus 1 is lowered.

### Method of Manufacturing an Image Pickup Apparatus

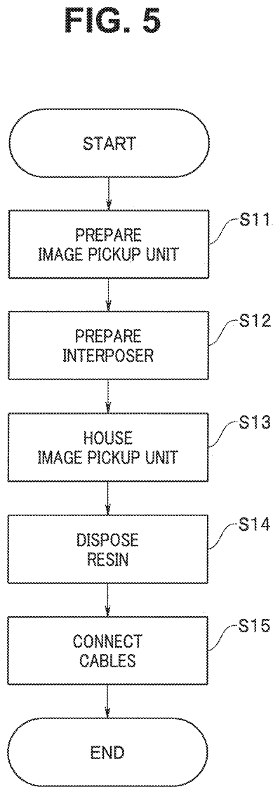

A method of manufacturing the image pickup apparatus 1 disposed on the distal end portion of the endoscope is simply described in accordance with a flowchart shown in FIG. 5.

### <Step S11> Image Pickup Unit Preparation Step

The image pickup unit is prepared by a wafer leveling method where a bond wafer formed by stacking a plurality of device wafers is cut.

An image pickup device wafer which includes the plurality of image pickup devices 11, and a plurality of semiconductor device wafers each including the plurality of semiconductor devices 21 to 23 are prepared.

For example, the plurality of light receiving portions 11A and the like are disposed on the image pickup device wafer by applying a known semiconductor manufacturing technique to a silicon wafer or the like. A peripheral circuit which applies primary processing of an output signal of the light receiving portion 11A and processes a drive control signal may be formed on the image pickup device wafer. It is preferable that a cover glass wafer which protects the light receiving portions 11A adhere to the image pickup device wafer before the penetrating wires 11H are formed in the image pickup device wafer from a rear surface

Then, the image pickup device wafer to which the cover glass wafer adheres using an adhesive layer (not shown) and solder bonding portions of the plurality of semiconductor device wafers each including the semiconductor devices 21 to 23 are bonded to each other, and the image pickup device wafer and the plurality of semiconductor device wafers are stacked to each other with sealing resins 25 to 27 sandwiched therebetween thus preparing the bonding wafer. The sealing resins 25 to 27 may be injected from a side surface of the bonding wafer after the image pickup device wafer and the plurality of semiconductor device wafers are bonded to each other, or disposed at the time of stacking the image pickup device wafer and the plurality of semiconductor device wafers.

The bonding wafer is cut so as to form individual rectangular parallelepiped image pickup units 10. In such an image pickup unit 10, four sides of the approximately rectangular light receiving portion 11A of the image pickup device 11 are respectively arranged parallel to four sides of an outer periphery of the image pickup device 11. Four side surfaces 10SS of the image pickup unit 10 prepared by a wafer leveling method are surfaces formed by cutting. Cutting may be performed by blade dicing in general. However, laser dicing or plasma dicing may be adopted.

In accordance with the above-mentioned step, the image pickup unit 10 is prepared where the image pickup unit 10 has the front surface 10SA and the rear surface 10SB on a side opposite to the front surface 10SA, and includes the cover glass 12, the image pickup device 11, and the stacked element 20 in which the plurality of semiconductor devices 21 to 23 are stacked and the rear electrodes 20P are disposed on the rear surface 10SB.

Corner portions of the image pickup unit 10 which are parallel to an optical axis O after cutting may be chamfered thus forming a cross-section in a direction orthogonal to the optical axis into a hexagonal shape, or the corner portions may be formed into a curved shape.

In other words, although the image pickup unit 10 is a rectangular parallelepiped body, the term “rectangular parallelepiped body” in the description of the present invention also includes an approximately rectangular parallelepiped body where corner portions are chamfered or formed into a curved surface.

### <Step S12> Interposer Preparing Step

The interposer 30 is prepared where the recessed portion C30 having the inner size larger than the outer size of the image pickup unit 10 is formed in the interposer 30, the connection electrodes 31 are disposed on the bottom surface C30SB of the recessed portion C30, and the bonding electrodes 32 connected with the connection electrodes 31 are disposed on the outer surface 30SB disposed opposite to the bottom surface C30SB.

The interposer 30 is formed of an MID (molded interconnect device) on which a conductive pattern is formed, for example. The interposer 30 may be formed of a ceramic stereoscopic wiring board. Further, although the interposer 30 is a rectangular parallelepiped body, the interposer 30 may have a circular cylindrical shape.

The order of the image pickup unit preparing step and the interposer preparing step may be reversed.

### <Step S13> Image Pickup Unit Housing Step

The image pickup unit 10 is inserted into the recessed portion C30 of the interposer 30, and the rear electrodes 20P and the connection electrodes 31 are bonded to each other. The inner size of the recessed portion C30 in the direction orthogonal to the optical axis is larger than the outer size of the image pickup unit 10 in the direction orthogonal to the optical axis. With such a configuration, a gap is formed between the image pickup unit 10 and the interposer 30.

The bonding portions formed between the rear electrodes 20P and the connection electrodes 31 are, for example, solder bonding portions, ultrasonic bonding portions, or heat ultrasonic bonding portions formed by applying heat in addition to ultrasonic waves.

To facilitate bonding between the rear electrodes 20P and the connection electrodes 31, it is preferable that a depth of the recessed portion C30 be set in a state that a portion of the image pickup unit 10, for example, a portion of the cover glass 12 protrudes from the recessed portion C30, and the protruding portion of the image pickup unit 10 be held by a jig at the time of bonding.

### <Step S14> Resin Disposing Step

The sealing resin 35 is disposed in the gap formed between the image pickup unit 10 and the interposer 30. For example, the sealing resin 35 in an uncured and liquid state is injected in the gap, and the sealing resin 35 is cured and solidified by heat treatment at a temperature of approximately 100° C. The sealing resin 35 is a resin such as an epoxy resin or a silicone resin having excellent moisture resistance and having substantially the same thermal expansion coefficient as the semiconductor device 21. The sealing resin 35 may be the same resin as the sealing resins 25 to 27.

A through hole through which the sealing resin 35 is injected into the gap may he formed on the bottom surface C30SB or the wall surface 30SS of the recessed portion C30 of the interposer 30.

### <Step S15> Cable Connecting Step

The bonding electrodes 32 of the interposer 30 and the electric cables 40 are bonded to each other. The distal end portions of the electric cables 40 are inserted into and bonded by soldering to the holes H30 of the interposer 30.

Since the electric cables 40 are inserted into the holes H30, the electric cables 40 can be easily positioned, and the positional displacement of the electric cables 40 minimally occurs after the electric cables 40 are inserted. Accordingly, such a bonding operation can be performed easily.

In the cable connecting step, it is necessary to hold and fix an outer peripheral surface of the image pickup unit 10 by a jig. In the image pickup apparatus 1, the image pickup unit 10 is housed in the interposer 30 and hence, there is no risk that the image pickup unit 10 is broken at the time of bonding the cables. Further, an arrangement interval between the bonding electrodes 32 of the interposer 30 can be set larger than an arrangement interval (inter-electrode pitch) between the plurality of rear electrodes 20P of the image pickup unit 10 and hence, a bonding operation of the electric cables 40 can be performed easily.

As has been described above, the method of manufacturing an. image pickup apparatus according to the present embodiment is a method of manufacturing a small-sized and highly reliable image pickup apparatus.

### Second Embodiment

An image pickup apparatus 1A, a method of manufacturing an image pickup apparatus, and an endoscope (hereinafter referred to as “image pickup apparatus or the like”) according to the second embodiment are similar to the image pickup apparatus 1 and the like according to the first embodiment and hence, the image pickup apparatus and the like according to the second embodiment have substantially the same advantageous effects as the image pickup apparatus 1 and the like of the first embodiment. Accordingly, constitutional components having the same functions as the corresponding constitutional components in the first embodiment are given with identical symbols, and the description of these constitutional components is omitted.

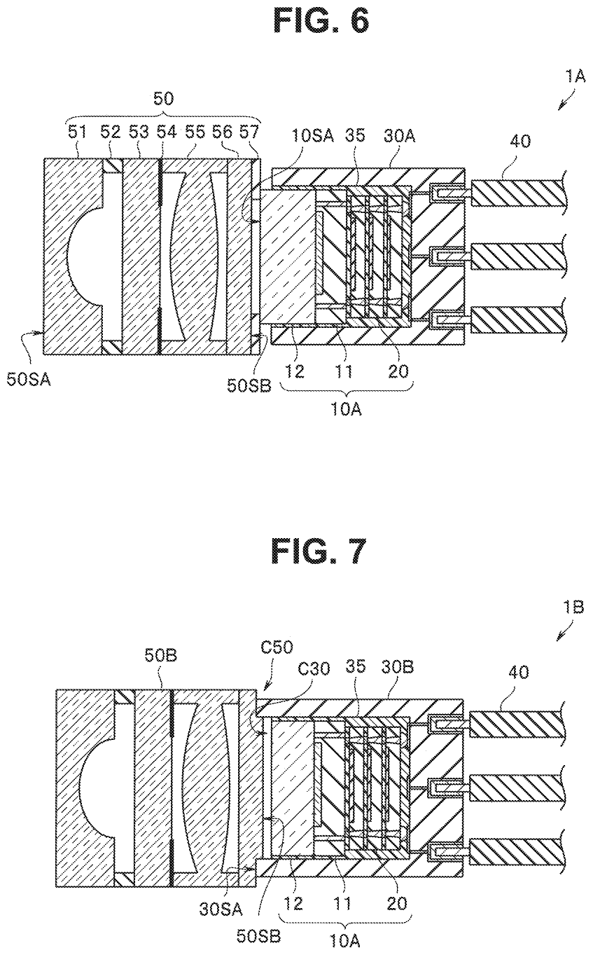

As shown in FIG. 6, the image pickup apparatus 1A further includes an optical unit 50 formed by stacking optical members 51 to 57 in addition to the configuration of the image pickup apparatus 1. The optical unit 50 has an incident surface 50A on which light is incident, and an emitting surface 50SB disposed on a side opposite to the incident surface 50SA. The emitting surface 50SB is fixed to an image pickup unit 10A by an adhesive layer (not shown) in a state where the emitting surface 50SB opposedly faces a front surface 10SA of the image pickup unit 10A.

The optical members 51, 55 are lenses, the optical members 52, 57 are spacers, the optical member 53 is a filter, the optical member 56 is a protective glass, and the optical member 54 is an aperture. The number of optical members, the arrangement of the optical members and the like are set corresponding to the specification of the optical unit.

The optical unit 50 is, for example, as in the case of the image pickup unit 10, prepared by a wafer leveling method where a stacked optical member wafer which is formed by stacking a plurality of optical member wafers each including a plurality of optical members respectively is cut. The optical unit 50 prepared by the wafer leveling method is a rectangular parallelepiped body, and side surfaces of the optical unit 50 are cut sections.

In the image pickup apparatus 1A, the image pickup unit 10A which is housed in and protected by an interposer 30A is integrally formed with the optical unit 50 and hence, productivity of the image pick-up apparatus 1A is high.

In the image pickup unit 10A, a size (an outer size in the direction orthogonal to an optical axis) of a cover glass 12 and the image pickup device 11 differ from a size of a stacked element 20.

The optical unit 50 and the image pickup unit 10A may be prepared by a wafer leveling method simultaneously. In other words, a plurality of optical member wafers each including a plurality of optical members and a plurality of semiconductor device wafers each including a plurality of semiconductor devices are stacked, and the image pickup units each equipped with the optical unit are prepared by cutting the stacked body. The optical unit 50 and the image pickup unit 10A prepared by the above-mentioned method have the same size.

### Modifications of Second Embodiment

Image pickup apparatuses and the like according to modifications of the second embodiment are similar to the image pickup apparatus 1A and the like and hence, the image pickup apparatuses and the like according to the modifications of the second embodiment have substantially the same advantageous effects as the image pickup apparatus 1A. Accordingly, constitutional components having the same functions as the corresponding constitutional components in the second embodiment are given with identical symbols, and the description of these constitutional components is omitted.

### Modification 1 of Second Embodiment

As shown in FIG. 7, in an image pickup apparatus 1B according a modification 1 of the second embodiment, a cutout C50 is formed on an emitting surface 50SB of an optical unit 50B. A front portion (a front surface 30SA and an inner surface) of an interposer 30B is brought into contact and engages with the cutout C50 by fitting.

In the image pickup apparatus 1B, a cover glass 12 of an image pickup unit 10A is completely housed in a recessed portion C30 of the interposer 30B. Accordingly, the image pickup unit 10A exhibits higher impact resistance than the image pickup unit 10. Further, in the image pickup apparatus 1B, positioning (optical axis alignment) between the optical unit 50B and the image pickup unit 10A can be performed easily.

The image pickup apparatus 1B exhibits higher reliability than the image pickup apparatus 1A and can be manufactured easily.

### Modification 2 of Second Embodiment

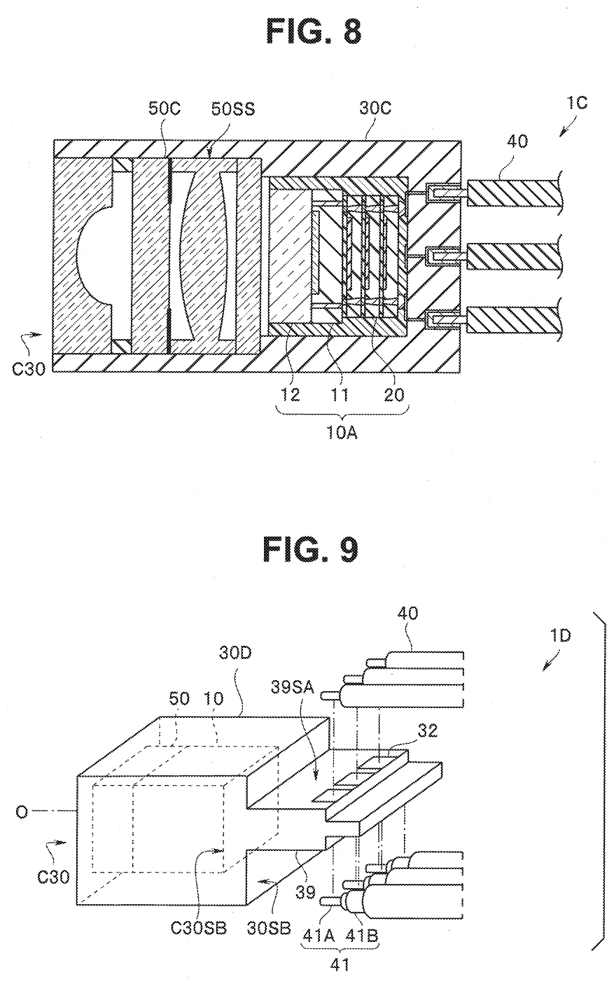

As shown in FIG. 8, in an image pickup apparatus 1C according to a modification 2 of the second embodiment, an optical unit 50C is also housed in a recessed portion C30 of an interposer 30C.

Accordingly, the image pickup apparatus 1C exhibits higher impact resistance than the image pickup apparatus 1A.

### Modification 3 of Second Embodiment

As shown in FIG. 9, an interposer 30D of an image pickup apparatus 1D of the modification 3 includes an extension region 39 which extends from an outer surface 30SB disposed opposite to a bottom surface C30SB of a recessed portion C30 and has a bonding surface 39SA parallel to an optical axis O. Bonding electrodes 32 are disposed on the bonding surface 39SA.

Electric cables 41 are bonded to a surface of the extension region 39 disposed opposite to the bonding surface 39SA. Each electric cable 41 has a core 41A and a shield line 41B. A stepped portion is formed on the extension region 39, and electrodes to which the shield lines 41B are bonded are disposed on the stepped portion (not shown).

### Modifications 4, 5 of Second Embodiment

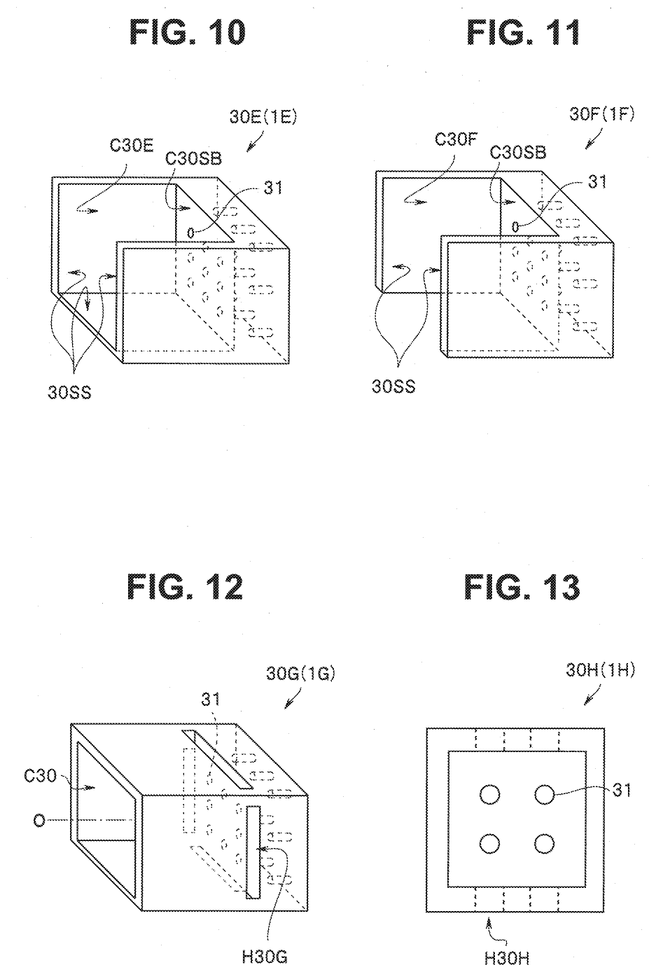

As shown in FIG. 10, in a modification 4 of the second embodiment, a recessed portion C30E of an interposer 30E of an image pickup apparatus 1E has three wall surfaces 30SS which opposedly face three side surfaces of an image pickup unit 10.

As shown in FIG. 11, in a modification 5 of the second embodiment, a recessed portion C30F of an interposer 30F of an image pickup apparatus 1F has two wall surfaces 30SS which opposedly face two side surfaces of an image pickup unit 10.

In other words, the image pickup unit is protected provided that the recessed portion of the interposer has two wall surfaces which opposedly face at least two side surfaces of the image pickup unit. It is preferable that side surfaces of the image pickup unit (and an optical unit) which are not covered by the interposer be covered by a sealing resin 35. Note that connection electrodes 31 are disposed on a bottom surface C30SB.

However, it is more preferable that, as in the case of the image pickup apparatuses 1 and 1A to 1D, the recessed portion of the interposer have four wall surfaces which opposedly face four side surfaces of the image pickup unit (and the optical unit) from a view point of a mechanical strength, moisture resistance, and light shielding property.

### Modifications 6, 7 of Second Embodiment

As shown in FIG. 12, in a modification 6 of the second embodiment, four side surface through holes H30G are formed in an interposer 30G of an image pickup apparatus 1G at positions on extensions of bonding portions which are formed of rear electrodes and connection electrodes 31 in a direction orthogonal to an optical axis.

In the image pickup apparatus 1G, the bonding portions can be observed from the side surface through holes H30G after an image pickup unit is bonded to the interposer 30G. When it is determined that bonding is not sufficient, bonding treatment is performed again immediately. In the image pickup apparatus 1G, a sealing resin 35 can be injected through the side surface through holes H30G. In injecting the sealing resin 35, the sealing resin 35 is also disposed in the side surface through holes H30G.

The image pickup apparatus 1G exhibits higher productivity than the image pickup apparatus 1 or the like.

The positions and the number of side surface through holes formed for observation of the bonding portions are designed corresponding to the positions and the number of bonding portions. For example, in a modification 7 of the second embodiment shown in FIG. 13, an interposer 30H of an image pickup apparatus 1H has four connection electrodes 31 and four side surface through holes H30H.

### Modification 8 of Second Embodiment

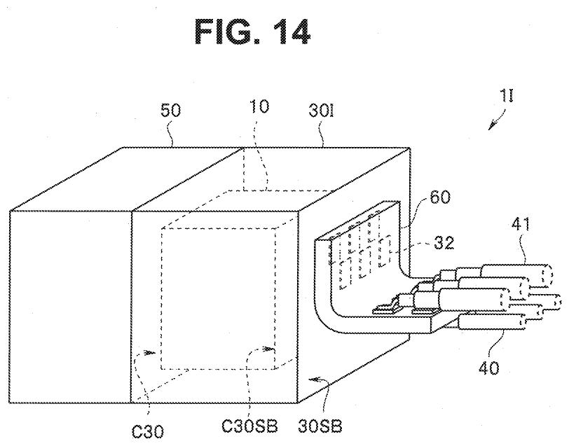

As has been described previously, in the image pickup apparatuses of the embodiments, an object to which the bonding electrodes 32 of the interposer are bonded may be a wiring board. As shown in FIG. 14, an image pickup apparatus 1I according to a modification 8 of the second embodiment further includes a wiring board 60. For example, electrodes (not shown) of the flexible wiring board 60 having a substrate made of polyimide are bonded to bonding electrodes 32 disposed on an outer surface 30SB of an interposer 301 disposed opposite to a bottom surface C30SB of a recessed portion C30. Electric cables 40, 41 are bonded to the wiring board 60. In an image pickup apparatus having an interposer 30D as in the case of the image pickup apparatus 1D of the modification 3, a wiring board 60 may be bonded to an extension region 39.

Needless to say, endoscopes 9A to 9I having the image pickup apparatus 1A according to the embodiment 2 and the image pickup apparatuses 1B to 1I according to the modifications of the second embodiment have the advantageous effects of the respective image pickup apparatuses 1A to 1I in addition to advantageous effects of the endoscope 9.

The present invention is not limited to the above-mentioned embodiments and the like, and the present invention can be altered or modified without departing from the gist of the present invention.

What is claimed is:

1. An image pickup apparatus disposed in an endoscope, the image pickup apparatus comprising:
an image pickup unit including an image pickup device, a stacked element formed by stacking a plurality of semiconductor devices, and a plurality of rear electrodes;
an interposer having a recessed portion in which the image pickup unit is housed, wherein a plurality of connection electrodes which are respectively connected with the plurality of rear electrodes are disposed on a bottom surface of the recessed portion, and a plurality of bonding electrodes which are respectively connected with the plurality of connection electrodes are disposed on an outer surface of the interposer;
a sealing resin disposed between the image pickup unit and the interposer; and
a plurality of electric cables or a wiring hoard respectively connected to the plurality of bonding electrodes of the interposer, wherein
an arrangement interval between the plurality of bonding electrodes is set larger than an arrangement interval between the plurality of connection electrodes.

2. The image pickup apparatus according to claim 1, wherein
the image pickup apparatus includes the plurality of electric cables,
the image pickup unit includes a cover glass which protects a light receiving portion of the image pickup device,
a portion of the cover glass protrudes from the recessed portion of the interposer,
the recessed portion of the interposer has four wall surfaces which opposedly face four side surfaces of the image pickup unit, and
a plurality of holes are formed in the outer surface of the interposer, and distal end portions of the plurality of electric cables are respectively inserted into the plurality of holes on each of which a bonding electrode is disposed.

3. The image pickup apparatus according to claim 1, wherein the recessed portion of the interposer includes four wall surfaces which opposedly face four side surfaces of the image pickup unit.

4. The image pickup apparatus according to claim 1, wherein a portion of the image pickup unit protrudes from the recessed portion of the interposer.

5. The image pickup apparatus according to claim 4, wherein
the image pickup unit includes a cover glass which protects a light receiving portion of the image pickup device, and
a portion of the cover glass protrudes from the recessed portion of the interposer.

6. The image pickup apparatus according to claim 1, further comprising an optical unit having an incident surface on which light is incident and an emitting surface from which the light incident on the incident surface is emitted, the optical unit being disposed such that the emitting surface opposedly faces a light receiving portion of the image pickup device, and being formed by stacking a plurality of optical members.

7. The image pickup apparatus according to claim 6, wherein an outer size of the image pickup device in a direction orthogonal to an optical axis differs from an outer size of the stacked element in the direction orthogonal to the optical axis.

8. The image pickup apparatus according to claim 1, wherein
the image pickup apparatus includes the plurality of electric cables, and
a plurality of holes are formed in the outer surface of the interposer, and distal end portions of the plurality of electric cables are respectively inserted into the plurality of holes on each of which a bonding electrode is disposed.

9. The image pickup apparatus according to claim 1, wherein the interposer includes an extension region extending from the outer surface, and having a bonding surface parallel to an optical axis, and
the plurality of bonding electrodes are disposed on the bonding surface.

10. The image pickup apparatus according to claim 6, wherein the optical unit is housed in the recessed portion of the interposer.

11. The image pickup apparatus according to claim 6, wherein a cutout is formed in the emitting surface of the optical unit, and
a front surface of the interposer is brought into contact and engages with the cutout by fitting.

12. The image pickup apparatus according to claim 11, wherein
the cutout and the front surface of the interposer are formed at a position where an optical axis of the optical unit and an optical axis of the image pickup unit are aligned with each other.

13. The image pickup apparatus according to claim 1, wherein a plurality of side surface through holes are formed in the interposer at positions disposed on extensions of a plurality of bonding portions which are formed of the respective plurality of rear electrodes and the respective plurality of connection electrodes in a direction orthogonal to an optical axis, and
the sealing resin is also disposed in the side surface through holes.

14. An endoscope comprising an image pickup apparatus, wherein the image pickup apparatus includes:
an image pickup unit including an image pickup device, a stacked element formed by stacking a plurality of semiconductor devices, and a plurality of rear electrodes;
an interposer having a recessed portion in which the image pickup unit is housed, wherein a plurality of connection electrodes which are respectively connected with the plurality of rear electrodes are disposed on a bottom surface of the recessed portion, and a plurality of bonding electrodes which are respectively connected with the plurality of connection electrodes are disposed on an outer surface of the interposer;
a sealing resin disposed between the image pickup unit and the interposer; and
a plurality of electric cables or a wiring board respectively connected to the plurality of bonding electrodes of the interposer, wherein
an arrangement interval between the plurality of bonding electrodes is set larger than an arrangement interval of the plurality of the connection electrodes.

15. A method of manufacturing an image pickup apparatus disposed in an endoscope, the method comprising:
preparing an image pickup unit including an image pickup device, a stacked element formed by stacking a plurality of semiconductor devices, and a plurality of rear electrodes;
preparing an interposer having a recessed portion, wherein a plurality of connection electrodes are disposed on a bottom surface of the recessed portion, and a plurality of bonding electrodes which are respectively connected with the plurality of connection electrodes are disposed on an outer surface of the interposer;
housing the image pickup unit in the recessed portion of the interposer, and connecting the respective plurality of rear electrodes with the respective plurality of connection electrodes;
disposing a resin between the image pickup unit and the interposer; and
connecting a respective plurality of electric cables or a wiring board to the plurality of bonding electrodes of the interposer, wherein
an arrangement interval between the plurality of bonding electrodes is set larger than an arrangement interval between the plurality of connection electrodes.
